(19) United States
(12) Patent Application Publication
(10) Pub. No.: US 2011/0009758 A1
(43) Pub. Date: Jan. 13, 2011

(54) SYSTEM AND METHOD FOR HEART MONITORING

(75) Inventor: Charles L. Richardson, Monroe, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: LifeScience Solutions LLC

(21) Appl. No.: 12/613,140

(22) Filed: Nov. 5, 2009

Related U.S. Application Data
(60) Provisional application No. 61/224,687, filed on Jul. 10, 2009.

(51) Int. Cl. A61B 5/0402

(52) U.S. Cl. 600/509

(57) ABSTRACT

Disclosed are systems and methods for monitoring a heart. According to one embodiment, the system includes a registering unit positioned external to the patient's body. The registering unit comprises a first controller configured to register an electrical signal from the heart. The system includes a second controller in operable communication with the first controller. The second controller is configured to receive the data from the first controller corresponding to the registered electrical signal and to compare the registered electrical signal to a baseline electrical signal to determine whether the heart is functioning properly.

### CROSS-REFERENCE TO RELATED APPLICATIONS

The present application claims priority to U.S. Provisional Patent Application Ser. No. 60/111,856, filed Nov. 6, 2008 and U.S. Provisional Patent Application Ser. No. 61/224,687, filed Jul. 10, 2009, each of which is incorporated by reference in its entirety.

### FIELD

Disclosed are systems and methods for monitoring and evaluating cardiac function and, more particularly, to non-invasive systems and methods for monitoring and evaluating the cardiac function of heart transplant and congestive heart failure patients, detecting heart failure in such patients, and providing an appropriate warning to the patient and/or physician in the event of actual or anticipated heart failure, and/or administering therapeutic drugs to the patient to treat the patient's condition.

### BACKGROUND

Cardiovascular disease if the leading cause of death for both men and women in the U.S. today and claims more lives each year than the next five leading causes of death combined.

In the United States, nearly 5 million patients have been diagnosed with heart failure. Each year more than 500,000 new cases are recognized. This represents, by far the fastest growing area of cardiology. As many as 20% of these patients qualify for an implanted device, either an implantable pacemaker or implantable cardiac defibrillator (“ICD”) or a biventricular pacemaker/ICD, and a fortunate percent of those severely symptomatic individuals will go on to cardiac transplant.

The primary diagnoses associated with heart transplantation are coronary artery disease (45%) and cardiomyopathy (45%), with congenital heart disease accounting for 8% and approximately 3% for retransplantation.

Each year approximately 2,500 cardiac transplants are performed in the United States and this number approaches 5,000 worldwide. One-year survival is approximately 85% in experienced transplant centers, with a five-year survival rate approaching approximately 70%. The most common cause of death is infection, followed by acute rejection. Although technology exists to treat bradycardia and tachycardia, i.e., pacemakers and defibrillators, respectively, the currently available apparatus and methods for monitoring a transplanted heart or for assisting in congestive heart failure assessment are quite limited and, for the most part, require the patient to undergo extensive invasive procedures or repetitive visits to a hospital or other medical facility which can be expensive.

Known methods for monitoring patients who receive a heart transplant generally involve an invasive procedure called endomyocardial biopsy (“EMB”). EMB procedures typically require an invasive biopsy of the transplanted heart in which the patient is taken to a catheterization laboratory and a large blood vessel (usually in the neck) is cannulated allowing a biopsy catheter to be advanced into the right side of the heart. Several small pieces or bites of the myocardium are sampled during the EMB, which are then sent for pathological evaluation. Similar invasive procedures are required of patients suffering from congestive heart failure, including catheterization to evaluate pressures inside the heart.

As discussed above, the rejection of a transplanted heart by the patient's body is one of the leading causes of death during the first year following the transplant. In order to detect early rejection of a transplanted heart, multiple EMBs are performed at regular, predetermined intervals. The typical patient undergoes up to twenty (20) EMBs during the first year. After the first year, even patients who have not experienced a rejection episode continue to require periodic EMBs to insure normal function of the transplanted heart. Although EMBs detect rejection and allow treatment in order to prevent death of the transplant patient, EMBs themselves result in a substantial risk of bleeding, infection, cardiac perforation, and other morbidities including death. In addition, this catheterization procedure is not only costly, but also painful and inconvenient for the patient.

Medical practitioners have attempted to reduce the risks associated with EMBs by exploring alternative methods for predicting transplant rejection and/or complications from congestive heart failure. For example, during the last decade investigators in Europe focused on intramyocardial electrograms and immune system markers that had the potential for predicting ischemia as well as acute transplant rejection. In studies on canines evaluating data from four myocardial sites, it was found that analysis of the mean intramyocardial unipolar peak-to-peak R-wave amplitude had a sensitivity (i.e., an ability to identify rejection) and a specificity (i.e., percentage of false positives) sufficient for diagnosing most transplant rejection. It also was discovered that, as the number of myocardial leads increases (i.e., the number of myocardial sites monitored increases), the sensitivity of detecting transplant rejection also increased. Preliminary studies on humans were able to show a correlation between acute rejection episodes and the mean amplitude of the R-wave of the QRS complex.

Over the past fifteen years, more than one thousand prototype unipolar, peak-to-peak rejection monitors (“UPPRMs”) have been implanted in both adults and children. UPPRMs require two or more electrodes attached to the patient's heart that are structured to register QRS voltage. The amplitude measurement of the intramyocardial electrogram (“IMEG”) was used to predict rejection. Another technique includes using electric current to measure the impedance consisting substantially of the ohmic resistance and the capacitive reactance. The ohmic resistance depends substantially on the extracellular space of the tissue, whereas the capacitive reactance depends substantially on the properties of the cell membrane. As a result of ischemia of the tissue during a rejection reaction, intracellular edema with simultaneous shrinkage of the extracellular space occurs, which results in changes to the ohmic resistance and capacitive reactance of the tissue.

Results have suggested advantages of these alternative methods over current methods of transplant rejection assessment such as EMBs. In particular, UPPRMs enabled reliable recognition of transplant rejection episodes at an earlier stage, thus allowing prompt treatment to reverse rejection and to block further development to more severe stages. Because advanced stages of transplant rejection were not encountered, the amount of additional immuno-suppression necessary to terminate rejection was moderate thereby reducing the treatment costs. Compared to an eighty-five percent (85%) survival rate for one-year post transplant when EMBs are used to assess transplant rejection, there were no deaths from acute transplant rejection when UPPRMs were used to assess rejection, provided the patient adhered strictly to short-interval, and preferably daily, IMEG recording. Biopsy findings showed the IMEGs to have one hundred percent (100%) sensitivity and ninety-seven percent (97%) specificity in detecting transplant rejection and there were 3% false negatives. In those few cases when the UPPRMs indicated transplant rejection with negative biopsy results (reason for less than one hundred percent (100%) specificity), all of these patients went on to have transplant rejection within twenty-four (24) to forty-eight (48) hours.

However, simple IMEG amplitude measurement is subject to variation due to the patient's daily rhythm, exercise status, and medications. A drop in amplitude may not always correlate to a rejection reaction. Moreover, because conventional UPPRMs provide at best only periodic monitoring (i.e., only while the patient is sleeping) the IMEG data registered by the UPPRMs does not provide the best data for determining a rejection reaction.

In light of the foregoing, it would be desirable to provide methods and systems capable of eliminating the risks associated with EMBs while at the same time providing more comprehensive data regarding the function of a patient's heart. Specifically, the methods and systems should allow for continuous, non-invasive monitoring of a patient's heart to thereby accurately detect heart rejection or failure at an early phase. In addition, the methods and systems should enable medical personnel to obtain historic and real-time monitoring data and information about the patient's heart so that the medical personnel can more effectively diagnose, discuss, coordinate or alter the patient's treatment.

### BRIEF SUMMARY OF THE INVENTION

Embodiments of the present invention provide non-invasive systems and methods for monitoring and evaluating the cardiac function of heart transplant and congestive heart failure patients, detecting heart failure in such patients and providing an appropriate warning to the patient and/or physician in the event of actual or anticipated heart failure, and/or administering therapeutic drugs to the patient to treat the patient's condition.

According to certain embodiments, one system for monitoring a patient's heart includes a registering unit located external to the patient's body. The registering unit includes a first controller in electrical communication with the patient's heart. The first controller is structured to register electrical signals from the patient's heart, such as a baseline electrical signal and an electrical signal registered after the baseline electrical signal. The system includes a second controller in operable communication with the first controller of the registering unit and configured to receive the baseline electrical signal and the at least one registered electrical signal. The second controller may be located remotely from the first controller. The second controller is configured to generate: (i) a baseline template corresponding to the baseline electrical signal; and (ii) at least one second template corresponding to the at least one registered electrical signal, and wherein the second controller is configured to compare the at least one second template to the baseline template to determine whether the patient's heart is functioning properly.

According to additional aspects of the system, the system further includes a plurality of electrodes coupled to the registering unit and positioned external to the patient's body. The plurality of electrodes are configured to be in electrical communication with the patient's heart and communicate the baseline electrical signal and the at least one registered electrical signal to the first controller. In another aspect, the system further includes a relay unit in communication with the first controller of the registering unit, wherein the relay unit is configured to receive the baseline electrical signal and the at least one registered electrical signal from the first controller. The relay unit may be further configured to transmit instructions to the registering unit and to transmit the baseline electrical signal and the at least one registered electrical signal to the second controller.

The second controller may include a data repository configured to receive and store data (e.g., electrically or magnetically) corresponding to the electrical signals from the patient's heart. In one construction, the registering unit includes a transmitter in operable communication with the second controller and configured to transmit the baseline electrical signal and the at least one registered electrical signal to the second controller. In another construction, the registering unit is in at least one of electrical or optical communication with the second controller.

The second controller may be configured to measure the area between the first template and the second template to determine whether the patient's heart is functioning properly. In another construction, the second controller is structured to identify a plurality of comparison points for the first template and to identify a plurality of comparison points for the second template. Each of the plurality of comparison points for the second template corresponds to one of the comparison points for the first template. The second controller is further structured to measure differences between each of the corresponding plurality of comparison points for the first template and the second template to determine whether the patient's heart is functioning properly. The second controller may be configured to communicate the baseline template, the at least one second template, and the measured differences between the corresponding plurality of comparison points for the baseline template and the at least one second template to a remote monitoring center via a communications network.

Also disclosed are methods for monitoring a patient's heart. According to one embodiment, the method includes registering a baseline electrical signal from the patient's heart with a first controller in electrical communication with the patient's heart (and positioned external thereto) and registering at least one registered electrical signal after the registration of the baseline electrical signal. The method further includes communicating data corresponding to the registered baseline electrical signal and at least one registered electrical signal from the first controller to a second controller. Moreover, the method includes generating a baseline template corresponding with the registered baseline electrical signal and generating at least one second template corresponding with the at least one registered electrical signal. The method also includes comparing the at least one second template with the baseline template to determine whether the patient's heart is functioning properly.

In one embodiment, the area between the first template and the second template is measured to determine whether the patient's heart is functioning properly. In another embodiment, the comparing step includes identifying a plurality of comparison points for both the first template and second template. Each of the plurality of comparison points for the second template corresponds to one of the comparison points for the first template. The differences between each of the corresponding plurality of comparison points for the first template and the second template are then measured to determine whether the patient's heart is functioning properly.

According to additional aspects, the method includes storing the data representing the registered baseline signal, the at least one registered electrical signal, the baseline template, the at least one second template in a data repository associated with the second controller. The method may also include communicating the data to a remote monitoring center via a communications network. Moreover, the providing step may include providing a registering unit comprising a first controller in electrical communication with the patient's heart via a plurality of electrodes positioned external to the patient's body. The communicating step may include communicating the registered baseline electrical signal and the at least one registered electrical signal from the first controller to the second controller located remotely from the first controller. For example, the communicating step may include wireless communication.

Further aspects of the present invention provide computer program products for monitoring a patient's heart. The computer program product includes a computer-readable storage medium having computer-readable program code portions stored therein. According to one embodiment, the computer-readable program portions include executable portions for performing, for example, the aforementioned method for monitoring a patient's heart.

According to another embodiment of the present invention, a system for monitoring an electrically active tissue (e.g., heart) of a patient is provided. The system includes a registering unit located external to the patient's body, wherein the registering unit comprises a first controller in electrical communication with the tissue, and wherein the first controller is configured to register a plurality of electrical signals from the tissue. The system also includes a plurality of electrodes coupled to the registering unit and positioned external to the patient's body, wherein the plurality of electrodes are configured to be in electrical communication with the tissue and communicate the plurality of registered electrical signals to the first controller. Furthermore, the system includes a second controller in operable communication with the first controller of the registering unit, wherein the second controller receives the plurality of registered electrical signals from the first controller and generates: (i) a baseline template corresponding to at least one of the registered electrical signals; and (ii) at least one second template corresponding to at least one other of the registered electrical signals. The second controller is configured to compare the at least one second template to the baseline template to determine whether the tissue is functioning properly.

Thus, there is provided methods and systems capable of eliminating the risks associated with EMBs while at the same time providing more comprehensive data regarding the function of a patient's heart. These methods and systems allow for accurate, non-invasive monitoring of a patient's heart to thereby detect heart rejection or failure at its earliest phase. In addition, the methods and systems enable medical personnel to obtain historic and real-time monitoring data and information about the patient's heart so that the medical personnel can more effectively diagnose, discuss, coordinate, or alter the patient's treatment.

### BRIEF DESCRIPTION OF THE DRAWINGS

Having thus described the invention in general terms, reference will now be made to the accompanying drawings, which are not necessarily drawn to scale, and wherein:

FIG. 1 is a diagram illustrating a system for monitoring a patient's heart, according to one embodiment;

FIG. 2 is a block diagram illustrating a registering unit, according to one embodiment;

FIG. 3 is a diagram showing a digitized electrogram or template, according to one embodiment;

FIG. 4 is a diagram graphically illustrating a comparison of a first template, which corresponds to a registered electrical signal from a patient's heart, to a second template, which corresponds to a baseline electrical signal from the patient's heart, according to one embodiment;

FIG. 5 is a diagram graphically illustrating a comparison of a first template, which corresponds to a registered electrical signal from a patient's heart, to a second template, which corresponds to a baseline electrical signal from the patient's heart, according to one embodiment;

FIG. 6 is a flow chart illustrating a method of monitoring a patient's heart, according to one embodiment;

FIG. 7 is a diagram illustrating a system for monitoring a patient's heart according to an additional embodiment of the present invention; and

FIG. 8 is a diagram illustrating a system for monitoring a patient's heart according to a further embodiment of the present invention.

### DETAILED DESCRIPTION

The present invention now will be described more fully hereinafter with reference to the accompanying drawings, in which some, but not all embodiments of the invention are shown. Indeed, these embodiments may be embodied in many different forms and should not be construed as limited to the embodiments set forth herein; rather, these embodiments are provided so that this disclosure will satisfy applicable legal requirements. Like numbers refer to like elements throughout.

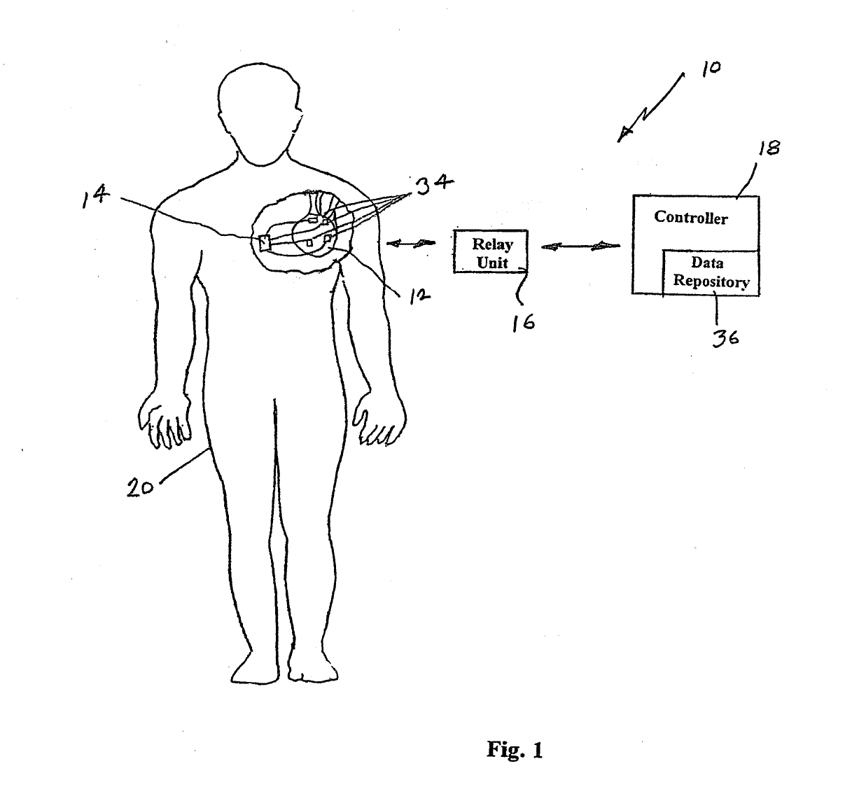

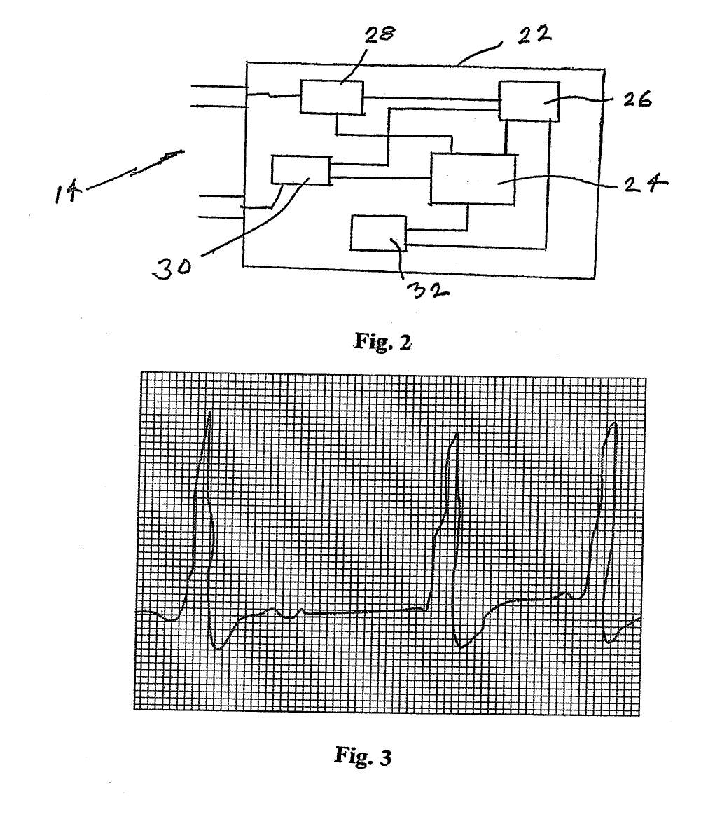

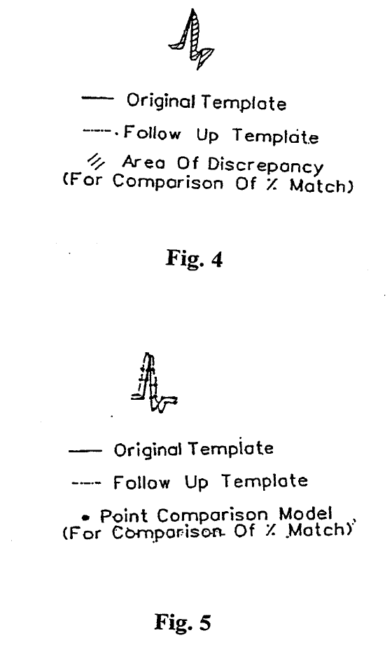

Referring to FIG. 1, there is illustrated a system 10 for monitoring the heart 12 of a heart transplant patient or a patient suffering congestive heart failure. The system 10 includes an implantable registering unit 14 for non-invasive monitoring of a patient's heart 12, a relay unit 16 for interrogating the registering unit, and a controller 18 for receiving data from the relay unit 16 corresponding to the patient's heart and analyzing the data. According to one embodiment, the registering unit 14 is structured to be implanted into the patient's body 20 and, thus, may include a housing 22 constructed of a relatively rigid material that is biologically inert, such as titanium and silicone. As illustrated in FIG. 2, the registering unit 14 can include a controller 24, such as a computer, microprocessor, or central processing unit operating under software control, an energy source 26, a receiver 30, and a transmitter/antenna 32. The registering unit 14 can optionally include a generator 28 structured to provide electrical stimulus to the patient's heart, a therapeutic process commonly referred to as “pacing.” The use of electrical stimuli to treat disorders such as bradyarrhythmias, or slow heart rhythms, and tachyarrhythmias, or fast heart rhythms, is well known to those skilled in the art and will not be further described herein. The energy source 26 is structured to provide electrical or thermal energy to the other components of the registering unit 14, including the controller 18, generator 28, receiver 30, and/or transmitter/antenna 32. The transmitter/antenna 32 is structured to communicate with the relay unit 16 electrically, such as through radio frequency communication, or optically. In one embodiment, the transmitter/antenna 32 includes an induction coil (not shown) that is structured to communicate with a corresponding induction coil (not shown) in the relay unit 16. Any commercially available pacemaker with appropriate software modifications can be used as the registering unit 14.

As illustrated in FIGS. 1 and 2, the controller 24 is in electrical communication with the patient's heart 12 via one or more sets or pairs of electrodes 34. According to one construction, as illustrated in FIG. 1, the system 10 includes two pairs of electrodes 34. The electrodes 34 can comprise any one of a number of commercially available epicardial (outside the surface of the heart) or endocardial (inside the heart) electrodes, as is well known to those skilled in the art. According to one embodiment, the electrodes 34 comprise screw-in epicardial bipolar IS I leads. The electrodes 34 preferably are attached to the heart 12 at the left and right ventricles, and left and right atriums. For example, one lead may be placed on the epicardial surface of the right or left atrium, one lead may be placed on the right ventricle, and two leads may be placed on the left ventricle. The lead placed over the right or left ventricle may be used to sense P-waves and to pace the atrium. Moreover, IMEG data may be recorded from any of the ventricular leads in bipolar or unipolar configurations. The electrodes 34 can be positioned at other areas about the patient's heart 12, depending on a variety of factors including, but not limited to, whether the patient is a heart transplant patient or suffering from congestive heart failure, the physical characteristics of the patients heart, or need for cardiac pacing. The electrodes 34 can be modified to include pressure sensors, which gauge vigor or degree of myocardial contraction.

The controller 18 can include a computer, microprocessor, or central processing unit operating under software control. As illustrated in FIG. 1, the controller 18 comprises a data depository 36 including hardware and associated software for data storage. The data repository 36 is in operable communication with the controller 18 via appropriate wiring or circuitry (not shown). The data repository 36 is structured to receive and store in computer-readable memory data corresponding to the electrical signals received from the patient's heart 12. The relay unit 16 is structured to transmit to the controller 18 data corresponding to the electrical signals received from the patient's heart 12 and to receive instructions transmitted by the controller 18 and transmit these instructions to the controller 24 of the registering unit 14 via the transmitter/antenna 32.

According to one embodiment, the controller 18 is located at the same location as the relay unit 16 and the patient, such as at a medical care facility or office or at the patient's home. For example, the relay unit 16 can be connected in operable communication with the controller 18 through a serial port connection or through a USB connection. According to another embodiment, the controller 18 is disposed remotely from the relay unit 16 and the patient. According to this construction, the relay unit 16 comprises another controller, such as a computer, microprocessor, or central processing unit operating under software control, that is in operable communication, preferably two-way operable communication, with the controller 18 via a communication network as is well known in the art. Such a communications network may comprise a land-line telephone network, a wireless network, a satellite communication network, or other suitable network to facilitate at least one-way, preferably two-way, communication between relay unit 16 and the controller 18. Advantageously, this later embodiment may eliminate the need to have an analog/digital converter or demodulator at the patient testing center.

Referring to FIGS. 1 and 2, when monitoring a patient's heart 12, the controller 18, either automatically at predetermined intervals or pursuant to instructions from an operator, communicates instructions to controller 24 of the registering unit 14 via the communication link between the relay unit 16 and transmitter/antenna 32 of the registering unit 14, instructing the registering unit to initiate monitoring of the patient's heart. The controller 24 then instructs the receiver 30 to begin registering or sensing the electrical signals emitted by the patient's heart. Data corresponding to the electrical signals registered by the receiver 30 is communicated to the controller 24. The controller 24 communicates the data representing the electrical signals registered by the receiver 30 to the transmitter/antenna 32, which then communicates the data to the relay unit 16. The relay unit 16 in turn communicates the data to the controller 18 for analysis.

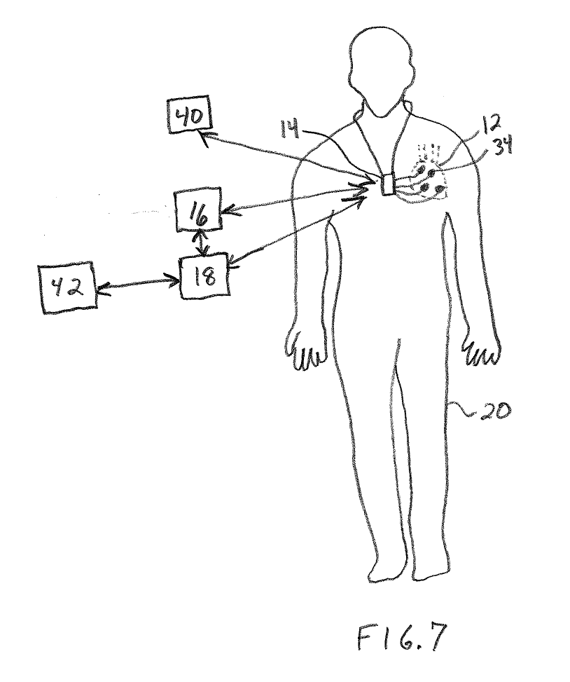

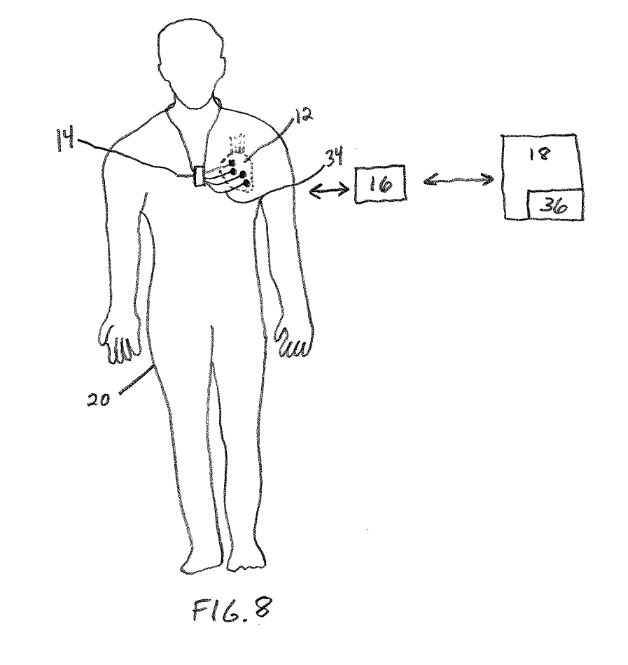

According to another embodiment, FIG. 7 illustrates that a physician interface unit 40 may also be provided. The physician unit 40 may comprises a controller and a relay unit similar to the relay unit 16 and controller 18 described above or other suitable communications link compatible with the registering unit 14. The physician interface unit 40 may be programmed with software enabling it to receive data from the registering unit 14 and display the data for review, for example, to show graphically in real time the data measured and transmitted by the registering unit 14. It may also be programmed to perform the data analysis described below. The physician interface unit 40 is also able to send instructions to the registering unit 14, for example, to change the value of programmable parameters of the registering unit 14 (such as a measurement interval), to interrogate the registering unit 14 for the actual values of the programmable parameters, or to command the registering unit 14 to transmit data or to begin or end pacing.

FIG. 7 also illustrates a data server 42 in communication with the controller 18 and configured to remotely receive data therefrom. The data server may include a data receiving software module in order to process the data with an analysis software module, which is capable of performing calculations, reference-waveform comparisons, and/or signal analysis described below. The processed data may be stored in a database, such as a structured query language (SQL) database. The data may then be accessed for users to view summaries of patient data, graphical analysis screens, and the like. The data may be accessed remotely, such as by a monitoring service at a remote computer, or by another authorized user, such as a patient's primary care physician, again at a remote computer, which communicates with the data server 42 by a network connection.

When monitoring a patient's heart 12, the registering unit 14 will be instructed, using either the relay unit 16 or the controller 18, to initiate monitoring of the patient's heart. The controller 24 begins registering or sensing the electrical signals emitted by the patient's heart. Data corresponding to the registered electrical signals is communicated to the transceiver 32, which then communicates the data to the controller 18 either directly or through the relay unit 16. Thus, the data corresponding to the registered electrical signals may be communicated directly to the controller 18 from the registering unit, thereby bypassing the relay unit 16.

The controller 18 and/or data server 42 operating under associated software control allow precise discrimination of intracellular and extracellular myocardial dynamics, as well as volume changes and myocardial strength of contraction in the patient's heart 12. The controller 18 and/or data server 42 are structured to analyze the data received from the patient's heart 12 in several ways. According to one procedure, each time the controller 18 and/or data server 42 receive data corresponding to the electrical signals received by the registering unit 14 from the patient's heart 12, the controller 18 and/or data server 42 digitally create or generate a graphical representation, such as a waveform, graph, or chart (referred to herein as a “template”), of a patient's intracardiac electrogram, such as the one illustrated in FIG. 3. For example, according to one form, the electrical signals received from the patient's heart comprise analog electrogram signals that are digitized by the controller 18 at 1 KHz with a 12-bit resolution and stored in the data repository 36 for later analysis. Preferably, a baseline electrical signal is registered using the above-referenced procedure to produce a template of the baseline electrical signal that is stored in the data repository 36 for later analysis. The baseline electrical signal can be obtained when the patient undergoes heart transplant, when the registering unit 14 is implanted, or at some other predetermined time.

The generation of the graphical representations by the controller 18 and/or data server 42 can include, but is not limited to: (1) individual beat identification (via peak detection algorithms); (2) Fourier decomposition of selected beats or beat sequences; (3) Fourier coefficient averaging and average signal reconstruction, assessing the average heart beat of the patient (as derived or computed from the data corresponding to the registered electrical signals received from the patient's heart 12, for critical time, area, derivative and amplitude markers using standard techniques); (4) utilizing Fourier coefficients and average waveform markers as descriptors of the graphical representation and comparing them using either time-series or auto- and cross-correlation analysis techniques; (5) constructing a modified wavelet template and performing a critical match percent correlation; (6) transforming wavelets, which are fragments of a complete waveform, to identify frequency and/or scale components of a signal simultaneously with their location in time; (7) using wavelet transformation that entails scale analysis via creation of mathematical structures that provide varying time/frequency/amplitude slices of a waveform for analysis.

Additionally, the registering unit 14 has the ability to measure resistance to current flow (impedance) from pacing stimuli given to the myocardium. The impedance is represented as a value in Ohms and is received in the data provided from the device. Multiple sets of data (unipolar and bipolar for each lead) will be received per data transmission. The impendence data is separate from the electrogram data.

The analysis by the controller 18 and/or data server 42 of the digitized electrograms or templates for the baseline electrical signal and the registered electrical signal involves comparing the templates to determine whether a predetermined critical match-percent threshold between the templates has been exceeded. For example, the analysis can include a modified wavelet template construction and a critical match percent correlation. Wavelet transformation (WT) identifies frequency or scale components of a signal and simultaneously with its location in time. The transformation entails scale analysis via creation of mathematical structures that provide varying time/frequency/amplitude slices for analysis. Each transformation is a fragment of a complete waveform and is termed as “wavelet.” Wavelets are optimal for approximating data with sharp discontinuities, such as myocardial electrograms. Since a percent wavelet match for a given heart rhythm is stable with regard to changes in body position and exercise over time, this algorithm offers greater sensitivity than conventional techniques, such as morphology algorithms (i.e., UPPRM), that analyze the amplitude of selected of a electrogram.

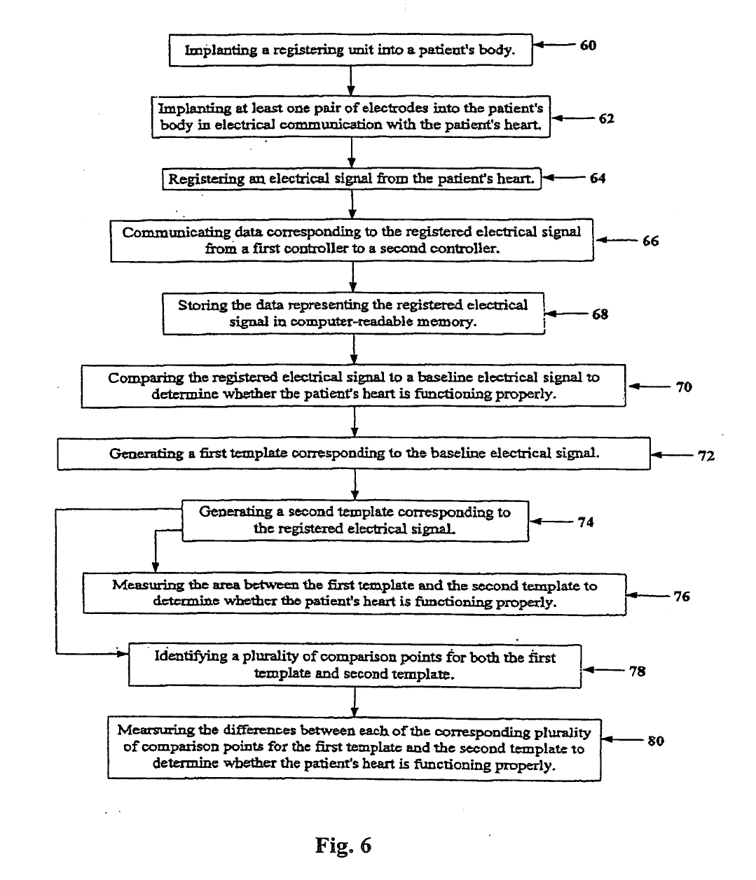

According to one embodiment, as illustrated in FIG. 5, the templates for the baseline electrical signal and the registered electrical signal are compared by measuring the area of discrepancy between the templates and determining a comparison percentage match, which can then be used to access whether the patient's heart is functioning properly. According to another embodiment, as illustrated in FIG. 6, the comparison involves identifying a plurality of comparison points for the baseline template and identifying a plurality of comparison points for the template corresponding to the registered electrical signal. Each of the plurality of comparison points for the second template corresponding to one of the comparison points for the baseline template. Thereafter, a correlation of points in the template will provide a comparison percentage match between the two templates, which can then be used to access whether the patient's heart is functioning properly.

According to one embodiment, one or more of the following parameters may be calculated for each lead vector and used in the analysis of the electric signals: area under the curve; area under dominant peak; area under minor peaks; base-to-dominant peak amplitude; peak-to-peak amplitude; nadir electrogram duration; slew rate of dominant peak upslope; slew rate of dominant peak downslope; and total electrogram duration. The average of each parameter may be calculated for each lead vector. Changes in one or more of the aforementioned parameters may be evaluated for assessing cardiac function using the baseline and registered templates.

A critical match-percentage threshold under either method (i.e., a discrepancy in the match points) of over thirty percent (30%), or more preferably, twenty (20%), or still more preferably, ten percent (10%) would indicate acute heart rejection. Thus, if a subsequent template corresponding to an electrical signal received from the patient's heart 12 does not correlate to the baseline template, by greater than seventy percent (70%), more preferably greater than eighty percent (80%), and still more preferably greater than ninety percent (90%), then rejection is present. Early detection of rejection advantageously permits prompt initiation of life saving therapy.

Also disclosed are methods for monitoring a patient's heart. According to one embodiment, the method includes implanting a registering unit into a patient's body. See Block 60. At least one pair of electrodes is implanted into the patient's body in electrical communication with the patient's heart. See Block 62. The method includes registering an electrical signal from the patient's heart. See Block 64. Data corresponding to the registered electrical signal is communicated from a first controller to a second controller. See Block 66. In one embodiment, the data representing the registered electrical signal is stored in computer-readable memory. See Block 68.

The method includes comparing the registered electrical signal to a baseline electrical signal to determine whether the patient's heart is functioning properly. See Block 70. In one embodiment, the comparing step includes generating a first template corresponding to the baseline electrical signal. See Block 72. A second template corresponding to the registered electrical signal is generated. See Block 74. Thereafter, the area between the first template and the second template is measured to determine whether the patient's heart is functioning properly. See Block 76. In another embodiment, the comparing step includes identifying a plurality of comparison points for both the first template and second template. See Block 78. Each of the plurality of comparison points for the second template corresponds to one of the comparison points for the first template. The differences between each of the corresponding plurality of comparison points for the first template and the second template are then measured to determine whether the patient's heart is functioning properly. See Block 80.

For additional exemplary techniques for monitoring and assessing cardiac function, see U.S. patent application Ser. Nos. 11/072,463 filed Mar. 7, 2005; 11/326,091 filed Jan. 5, 2006; 11/738,962 filed Apr. 23, 2007; and 11/871,524 filed Oct. 12, 2007, each of which is herein incorporated by reference in its entirety.

FIGS. 1, 2, and 6 are block diagrams, flowcharts and control flow illustrations of methods, systems and program products in certain embodiments. It will be understood that each block or step of the block diagrams, flowcharts and control flow illustrations, and combinations of blocks in the block diagrams, flowcharts and control flow illustrations, can be implemented by computer program instructions. These computer program instructions may be loaded onto a computer or other programmable apparatus to produce a machine, such that the instructions which execute on the computer or other programmable apparatus create means or devices for implementing the functions specified in the block diagrams, flowcharts or control flow block(s) or step(s). These computer program instructions may also be stored in a computer-readable memory that can direct a computer or other programmable apparatus to function in a particular manner, such that the instructions stored in the computer-readable memory produce an article of manufacture, including instruction means or devices which implement the functions specified in the block diagrams, flowcharts or control flow block(s) or step(s). The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions specified in the block diagrams, flowcharts or control flow block(s) or step(s).

Accordingly, blocks or steps of the block diagrams, flowcharts or control flow illustrations support combinations of means or devices for performing the specified functions, combinations of steps for performing the specified functions and program instruction means or devices for performing the specified functions. It will also be understood that each block or step of the block diagrams, flowcharts or control flow illustrations, and combinations of blocks or steps in the block diagrams, flowcharts or control flow illustrations, can be implemented by special purpose hardware-based computer systems which perform the specified functions or steps, or combinations of special purpose hardware and computer instructions.

The configuration described supra comprises an implanted registering unit 14 with a relay unit 16 and second controller 18 both located outside or external to the patient's body. Alternative configurations may also be employed as will now be described.

Initially, second controller 18, as described supra, digitally creates or generates a graphical representation of a patient's intracardiac electrogram. This graphical representation comprises a baseline template as well as one or more subsequently obtained registered electrical signal(s) which are compared and analyzed by the second controller 18, also as described supra. The second controller 18 may comprise, in certain embodiments, a computer having a keyboard for input and a display output and/or or a printing output that displays and/or is capable of printing the baseline template, one or more of the registered electrical signal templates corresponding to at least one registered electrical signals registered after registration of the baseline electrical signal corresponding with the baseline template, and/or the comparative results after analysis of the at least one registered electrical signal templates. These data and results may be graphic and/or narrative communication of the comparison between the baseline and one or more registered electrical signal templates which assist in determining whether the patient's heart is functioning properly. The second controller 18 comprising a computer may further transmit these data and results to a monitoring center (not shown in the figures) which is remotely located from the second controller 18. Such transmission may utilize a communication network as is well known in the art, such as a a land-line telephone network, a wireless network, a satellite communication network, or other suitable network to facilitate at least one-way, preferably two-way, communication with the monitoring center. In one embodiment, the monitoring center may be located within a health care facility such as, but not limited to, a hospital or the patient's doctor's office. Health care personnel within the health care facility may then remotely monitor the data and results coming into the monitoring center from the second controller 18 and may, in certain embodiments, communicate with, i.e., send and receive data and/or messages, the second controller 18 via the communications network.

In an alternative embodiment, the registering unit 14 may be disposed external to the patient's body and comprise a first controller 24 in operable communication with the electrical signals of the patient's heart via electrodes 34 also positioned external to the patient's body, as shown in FIG. 8. For example, FIG. 8 shows that the patient 20 may wear the registering unit on a lanyard, although the registering unit may be carried by the patient using various techniques, such as within or attached to the patient's clothing or attached to the patient with adhesives or bandages. Similarly, the electrodes may be attached to the patient's skin to ensure electrical communication with the patient's heart using various techniques such as adhesives or taping. In the embodiment shown in FIG. 8, it is not necessary that the registering unit 14 comprise a housing of biologically inert material since the registering unit is located external to the patient's body, although such construction is within the scope of this embodiment. With the exception of this embodiment comprising the registering unit 14 being located outside the patient's body, rather than implanted within the patient's body as in the embodiment's described supra, this embodiment (with an external registering unit 14) functions identically as all other embodiments described herein. Thus, the electrodes 34 need not be coupled directly to the patient's heart 12, which provides the added benefit of a less invasive procedure for monitoring the patient's heart.

Thus, embodiments of the present invention may provide techniques for effectively monitoring cardiac function. For example, embodiments of the present invention may provide the ability to predict the onset of the rejection of a transplanted heart or a myocardial infarction. In particular, studies have shown that embodiments of the present invention may detect the onset of grade 2 (values within 50-70% of baseline) and grade 3 (values less than 50% of baseline) rejections earlier than conventional biopsies. In some cases, even grade 1 rejections may be detected with ideal baseline templates. Furthermore, embodiments of the present invention may be able to detect, for instance, elevation or depression in ST waves, Q wave formation, and/or T wave changes in order to detect the onset of a myocardial infarction. Therefore, embodiments of the present invention provide for non-invasive techniques to effectively monitor and detect the onset of various heart complications, wherein such monitoring may occur remotely from the patient. An analysis of the electrical signals from the patient's heart may also reveal signs of damage to the heart, reveal problems with the electrical conduction system, assist in diagnosing a disease, and monitor the effects of drugs or devices such as pacemakers and implantable cardioverter defibrillators. In addition to the heart, embodiments of the present invention may be applicable to other electrically active tissues that generate detectable electrical signals, such as muscle and brain.

Many modifications and other embodiments of the invention set forth herein will come to mind to one skilled in the art to which this invention pertains having the benefit of the teachings presented in the foregoing descriptions and the associated drawings. Therefore, it is to be understood that the embodiments are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A system for monitoring a patient's heart, comprising:
a registering unit located external to the patient's body, wherein the registering unit comprises a first controller in electrical communication with the patient's heart, and wherein the first controller is configured to register from the patient's heart: (i) a baseline electrical signal; and (ii) at least one registered electrical signal after registration of the registered baseline electrical signal; and
a second controller in operable communication with the first controller of the registering unit, wherein the second controller receives the registered baseline electrical signal and the at least one registered electrical signal from the first controller and generates: (i) a baseline template corresponding to the baseline electrical signal; and (ii) at least one second template corresponding to the at least one registered electrical signal, and wherein the second controller is configured to compare the at least one second template to the baseline template to determine whether the patient's heart is functioning properly.

2. A system according to claim 1, further comprising a plurality of electrodes coupled to the registering unit and positioned external to the patient's body, wherein the plurality of electrodes are configured to be in electrical communication with the patient's heart and communicate the baseline electrical signal and the at least one registered electrical signal to the first controller.

3. A system according to claim 1, wherein the registering unit further comprises a transmitter in operable communication with the second controller and configured to transmit the baseline electrical signal and the at least one registered electrical signal to the second controller.

4. A system according to claim 1 wherein the registering unit is in at least one of electrical or optical communication with the second controller.

5. A system according to claim 1, wherein the second controller is located remotely from the registering unit.

6. A system according to claim 1, wherein the second controller comprises a data repository for storing data corresponding to the baseline electrical signal and the at least one registered electrical signal.

7. A system according to claim 1, further comprising a relay unit in communication with the first controller of the registering unit, wherein the relay unit is configured to receive data from the first controller corresponding to the baseline electrical signal and the at least one registered electrical signal.

8. A system according to claim 7, wherein the relay unit is further configured to transmit instructions to the registering unit and to transmit the baseline electrical signal and the at least one registered electrical signal to the second controller.

9. A system according to claim 1, wherein the second controller measures the area between the baseline template and the at least one second template to determine whether the patient's heart is functioning properly.

10. A system according to claim 1, wherein the second controller identifies a plurality of comparison points for the baseline template and identifies a plurality of comparison points for the at least one second template, each of the plurality of comparison points for the second template corresponding to one of the comparison points for the baseline template, and wherein the second controller identifies and measures differences between each of the corresponding plurality of comparison points for the baseline template and the at least one second template to determine whether the patient's heart is functioning properly.

11. A system according to claim 10, wherein the second controller is configured to communicate the baseline template, the at least one second template, and the measured differences between the corresponding plurality of comparison points for the baseline template and the at least one second template to a remote monitoring center via a communications network.

12. A method for monitoring a patient's heart, comprising:
registering a baseline electrical signal from the patient's heart with a first controller located external to the patient's body and in electrical communication with the patient's heart;
registering at least one registered electrical signal with the first controller after the registration of the baseline electrical signal;
communicating data corresponding to the registered baseline electrical signal and at least one registered electrical signal from the first controller to a second controller;
generating a baseline template corresponding with the registered baseline electrical signal;
generating at least one second template corresponding with the at least one registered electrical signal; and
comparing the at least one second template with the baseline template to determine whether the patient's heart is functioning properly.

13. A method according to claim 12, wherein the comparing step further comprises measuring the area between the baseline template and the at least one second template to determine whether the patient's heart is functioning properly.

14. A method according to claim 12, wherein the comparing step further comprises: identifying a plurality of comparison points for the baseline template; identifying a plurality of comparison points for the at least one second template, each of the plurality of comparison points for the at least one second template corresponding to one of the comparison points for the baseline template; and measuring differences between each of the corresponding plurality of comparison points for the baseline template and the at least one second template to determine whether the patient's heart is functioning properly.

15. A method according to claim 12, further comprising storing the data representing the registered baseline signal, the at least one registered electrical signal, the baseline template, the at least one second template in a data repository associated with the second controller.

16. A method according to claim 12, further comprising communicating the data to a remote monitoring center via a communications network.

17. A method according to claim 12, wherein the first controller is in electrical communication with the patient's heart via a plurality of electrodes positioned external to the patient's body.

18. A method according to claim 12, wherein communicating comprises communicating the registered baseline electrical signal and the at least one registered electrical signal from the first controller to the second controller located remotely from the first controller.

19. A method according to claim 18, wherein communicating comprises wirelessly communicating.

20. A system for monitoring an electrically active tissue of a patient, comprising:
a registering unit located external to the patient's body, wherein the registering unit comprises a first controller in electrical communication with the tissue, and wherein the first controller is configured to register a plurality of electrical signals from the tissue;
a plurality of electrodes coupled to the registering unit and positioned external to the patient's body, wherein the plurality of electrodes are configured to be in electrical communication with the tissue and communicate the plurality of registered electrical signals to the first controller; and
a second controller in operable communication with the first controller of the registering unit, wherein the second controller receives the plurality of registered electrical signals from the first controller and generates: (i) a baseline template corresponding to at least one of the registered electrical signals; and (ii) at least one second template corresponding to at least one other of the registered electrical signals, and wherein the second controller is configured to compare the at least one second template to the baseline template to determine whether the tissue is functioning properly.
